(19) United States
(12) Patent Application Publication
(10) Pub. No.: US 2025/0115879 A1
(43) Pub. Date: Apr. 10, 2025

(54) HYBRIDOMA CELL LINE SECRETING ANTI-POLYSTYRENE MONOCLONAL ANTIBODY AND USE THEREOF

(71) Applicant: Wuhan Textile University, Wuhan (CN)

(72) Inventors: Zhaowei Zhang, Wuhan (CN); Wenqin Wu, Wuhan (CN)

(21) Appl. No.: 18/966,182

(22) Filed: Dec. 3, 2024

(30) Foreign Application Priority Data
Oct. 15, 2024 (CN) 202411439989.6

(51) Int. Cl. C12N 5/12; C07K 16/44; G01N 33/44

(52) U.S. Cl. CPC C12N 5/12; C07K 16/44; G01N 33/442

(57) ABSTRACT

A hybridoma cell line secreting an anti-polystyrene monoclonal antibody and use thereof are provided. The hybridoma cell line includes hybridoma cell line PS-8 and hybridoma cell line PS-17 which are deposited with the China Center for Type Culture Collection under the Accession numbers of CCTCC NO: C2024144 and CCTCC NO: C2024154. The passage cell line can stably secrete the anti-polystyrene monoclonal antibody. The secreted antibody is high in potency, good in specificity and high in sensitivity, can be applied to kit detection, and has potential application values.

### SEQUENCE LISTING

The sequence listing is submitted as a XML file filed via EFS-Web, with a file name of “Sequence_Listing.XML”, a creation date of Nov. 30, 2024, and a size of 6077 bytes. The sequence Listing filed via EFS-Web is a part of the specification and is incorporated in its entirety by reference herein.

### TECHNICAL FIELD

The present disclosure belongs to the technical field of biological materials, and particularly relates to a hybridoma cell line secreting an anti-polystyrene monoclonal antibody and use thereof.

### BACKGROUND

Microplastics have many types, large hazards, heavy pollution and a wide range. Microplastics that pollute the environment mainly include polystyrene (PS), polypropylene (PP), polyethylene (PE), etc. Microplastics have hazards such as sublethal toxicity and cytotoxicity, posing a serious threat to human life and health.

Most of the existing microplastic detections depend on instrument analysis methods such as microscope spectroscopy and Raman spectroscopy, which is difficult to meet the needs of rapid and accurate detection. In China, microplastics are complicated in pollution environment, significant in geographical difference and multiple in pollution links, and therefore involved in the fields of environment, food, textiles and the like. In the process of producing synthetic fibers (such as polyester fibers and nylon), some plastic particles or fibers smaller than 5 mm may be produced. For example, friction of production equipment, incomplete polymerization of raw materials and other situations may lead to the generation of microplastics. In addition, in the textile process, such as spinning, weaving, printing and dyeing and other links, the operation of a machine, abrasion of parts and stress application during the processing may make microplastic fibers on the surface of the textile detach from the surface of the textile. Especially for some high-strength processing processes, poor raw materials are used, which may increase the possibility of generating microplastics. When people wear textiles, the fibers on the surface of the textile are gradually worn and fall off due to body movements, friction and others, thereby generating microplastic fibers. For example, fiber shedding is easier to occur in some areas frequently rubbed, such as cuffs, collars and pant legs. Meanwhile, during the washing in a washing machine, the friction between clothes, the impact of water flow and the action of detergent can cause a large number of fibers to fall off from the clothes, forming microplastic fibers. It is estimated that in a typical household washing activity, a piece of clothing can release millions of fibers. Furthermore, some clothes made of synthetic fibers, such as nylon and dacron, more easily produce microplastic fibers during the washing due to their fiber structures and features. When discarded textiles are buried, the textiles will gradually degrade and break in a natural environment over time to produce microplastics. These microplastics may enter environments such as soil and water with rainwash or other approaches, causing pollution. Although incineration can reduce the amount of solid waste from textiles, some smoke and ashes containing microplastics may be generated during the incineration. If these smoke and ashes are not treated well, they can also be released into the environment, causing microplastic pollution.

At present, the main detection methods of microplastics include visual inspection, microscopy, electron microscopy, Raman spectroscopy, infrared spectroscopy, thermal analysis, pyrolysis gas chromatography-mass spectrometry, laser infrared imaging and optical thermal infrared technology. However, some of these detection technologies can only make qualitative judgments, some detection technologies have low detection sensitivity, and some detection technologies rely on large-scale laboratory instruments for detection, thus the real-time monitoring for pollution distribution and environmental effects of the microplastics in the environment is difficult. Therefore, there is an urgent need to study high-sensitivity and rapid detection methods of microplastics in complex matrices. The key to a rapid quantitative analysis technology of microplastics is the development of microplastic-specific antibodies. At present, there is the development of microplastics (PS) polyclonal antibodies, but there is no preparation of microplastics (PS and other types of microplastics) monoclonal antibodies. Microplastics are inert small particles and therefore have significant challenges in the aspects of hapten preparation and measurement of parameters such as affinity.

Therefore, it is urgent to provide a hybridoma cell line secreting an anti-polystyrene monoclonal antibody and use thereof.

### SUMMARY

To solve the defects existing in the prior art, the present disclosure provides a hybridoma cell line secreting an anti-polystyrene monoclonal antibody and the use thereof.

To solve the above technical problem, the present disclosure provides the following technical solution:

The first objective of the present disclosure is to provide a hybridoma cell line secreting an anti-polystyrene monoclonal antibody, comprising hybridoma cell line PS-8 deposited with China Center for Type Culture Collection under the Accession number of CCTCC NO: C2024144.

The second objective of the present disclosure is to provide a hybridoma cell line secreting an anti-polystyrene monoclonal antibody, comprising hybridoma cell line PS-17 deposited with China Center for Type Culture Collection under the Accession number of CCTCC NO: C2024154.

The third objective of the present disclosure is to provide a monoclonal antibody, the monoclonal antibody being secreted by the hybridoma cell line PS-8 or the hybridoma cell line PS-17, and the monoclonal antibody specifically recognizing polystyrene.

The fourth objective of the present disclosure is to provide a kit, the kit comprising the monoclonal antibody secreted by the hybridoma cell line PS-8 or the hybridoma cell line PS-17.

The fifth objective of the present disclosure is to provide the use of the monoclonal antibody in preparing a reagent for detecting polystyrene.

Compared with the prior art, the present disclosure has the following beneficial effects: the passage cell line of the hybridoma cells provided by the present disclosure can stably secrete a monoclonal antibody against a polystyrene protein, and the secreted antibody is high in potency, good in specificity and high in sensitivity, and can be applied to kit detection, thereby significantly improving the sensitivity of kit detection. Collection of cells:

The hybridoma cell line secreting the anti-polystyrene monoclonal antibody provided by the present disclosure comprises hybridoma cell line PS-8 and hybridoma cell line PS-17, the hybridoma cell line PS-8 is screened by the inventor of the present disclosure, the hybridoma cell line PS-8 and the hybridoma cell line PS-17 are deposited with China Center for Type Culture Collection under the Accession numbers of CCTCC NO: C2024144 and CCTCC NO: C2024154 respectively on May 10, 2024, and the address of the collection unit is 299 Bayi Road, Wuchang District, Wuhan City, Hubei Province, on campus of Wuhan University.

### BRIEF DESCRIPTION OF THE DRAWINGS

FIG. 1 is a schematic diagram showing sodium dodecyl sulfate-polyacrylamide gel electrophoresis (SDS-PAGE) of PS-bovine serum albumin (BSA) and PS-keyhole limpet hemocyanin (KLH) in example 1 of the present disclosure;

FIG. 2A is a schematic diagram showing the reduced SDS-PAGE of each antibody in example 4 of the present disclosure;

FIG. 2B is a schematic diagram showing the non-reduced SDS-PAGE of each antibody in example 4 of the present disclosure;

wherein, the names of the antibodies corresponding to lanes 1-12 are PS-1, PS-3, PS-4, PS-7, PS-8, PS-9, PS-13, PS-15, PS-17, PS-18, PS-22 and PS-23.

FIG. 3 shows the control experimental results of each antibody coated with 5 different microplastics in example 4 of the present disclosure;

FIG. 4 shows an acquisition process of antibody variable regions in example 5 of the present disclosure; and

FIG. 5 is a standard curve graph in example 6 of the present disclosure.

### DETAILED DESCRIPTION OF THE EMBODIMENTS

Next, preferred embodiments of the present disclosure will be illustrated in combination with accompanying drawings, it should be understood that preferred embodiments described here are only used for illustrating and explaining the present disclosure, but not limiting the present disclosure.

### Example 1: Coupling of Microplastic Poly (Styrene, w-bromo-terminated (PS-CHOOH) and Coupling Protein Bovine Serum Albumin-keyhole Limpet Hemocyanin (BSA/KLH)

1. Microplastics, polylink coupling buffer, polylink ethyl-[3-(dimethylamino) propyl]-carbodiimide hydrochloride (EDAC) and polylink wash/storage buffer were cooled to a room temperature.

2. 12.5 mg of microplastics were added into a 1.5 mL centrifuge tube and then centrifuged for 5-10 min at 500-1000 g, and a supernatant was discarded; 0.4 mL of polylink coupling buffer was added for resuspension followed by centrifugation again, and then the supernatant was discarded; 0.17 mL of polylink coupling buffer was added again for resuspension.

3. 10 mg of polylink EDAC was dissolved into 50 μL of polylink coupling buffer to prepare a 200 mg/mL EDAC solution. Note: the solution was prepared when using.

4. 20 μL of EDAC solution was added into 0.17 mL of polylink coupling buffer suspension to be fixed on a rotator to be evenly mixed for 15 min at room temperature for activation.

5. 200-500 μg of coupling protein (BSA/KLH) was added and fixed on the rotator to be evenly mixed for 30-60 min at room temperature (the protein was dissolved with the polylink coupling buffer until 1-5 mg/mL).

6. The coupling protein (BSA/KLH) was centrifuged for 10 min at 500-1000 g, the supernatant was sucked away, and then the amount of the BSA/KLH was measured.

7. 0.4 mL of polylink wash/storage buffer was added for resuspension.

8. Steps 6 and 7 were repeated, and the precipitate at the bottom of the tube was PS-BSA or PS-KLH and finally stored at 4° C.

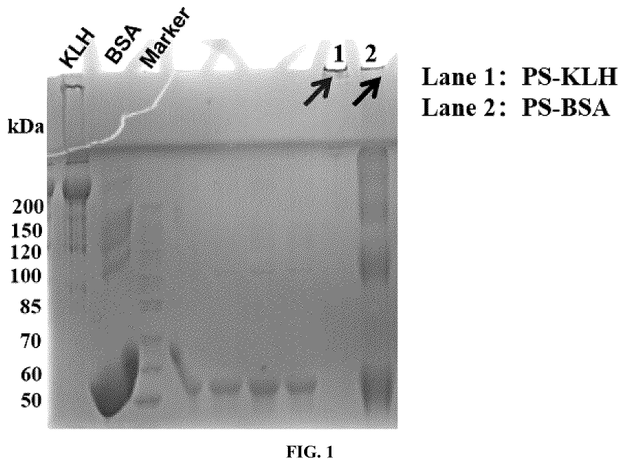

9. SDS-PAGE analysis (4% spacer gel and 7.5% separating gel) was performed. The detection results of reduced SDS-PAGE are shown in FIG. 1, proving that PS-KLH is successfully coupled with PS-BSA.

### Example 2: Preparation of Monoclonal Antibody

6 to 8-week-old female Balb/c mice were selected and immunologically injected with a prepared PS-BSA artificial antigen. Adult Balb/c mice received primary immunization through subcutaneous (in an area between shoulders) administration and were subjected to booster immunization every 2 weeks. Blood samples were collected at week 0 (before immunization), week 8 (2 weeks after the fourth booster jab), week 10 (before the third booster jab), week 12, and week 14, then serum isolation was performed to obtain B lymphocytes, and RNA was extracted and transcripted into a complementary DNA (cDNA) library. Amplification was performed again through cDNA to obtain a specific gene fragment, and the specific gene fragment was recombined onto a bacteriophage to achieve the construction of a nano antibody gene library. Subsequently, specific antibody screening was performed to obtain an antibody meeting requirements, and the PS-BSA artificial antigen was fixed on a carrier and then interacted with the bacteriophage library. Then, the surface of the carrier was washed to remove unbound or non-specific bound bacteriophages and then the binding of the vector was destroyed with a strong acid, thereby obtaining a positive bacteriophage solution. After 2-3 rounds were repeated, the monoclonal antibody meeting the requirements was obtained.

Through the injection of the PS-BSA artificial antigen, an immunization scheme for mice is shown in Table 1, the specific response of the PS-BSA artificial antigen on the immune system of Balb/c mice was observed, and an activation mechanism of an antibody-specific immune response was explored.

TABLE 1
Immunization scheme for mice

| | | | | | | Interval time |
|---|---|---|---|---|---|---|
| | | | | | | distanced |
| | | | | | | from the last |
| Method | Antigen | Route | Adjuvant | Dose (μg) | | immunization |
| The first | PS-BSA | Subcutaneous | Complete | 100 | μg/mouse | 0 |
| immunization | | | | | | |
| The second | PS-BSA | Subcutaneous | Incomplete | 50 | μg/mouse | 2 weeks |
| immunization | | | | | | |
| The third | PS-BSA | Subcutaneous | Incomplete | 50 | μg/mouse | 2 weeks |
| immunization | | | | | | |
| The fourth | PS-BSA | Subcutaneous | Incomplete | 50 | μg/mouse | 2 weeks |
| immunization | | | | | | |
| The fifth | PS-BSA | Subcutaneous | Incomplete | 50 | μg/mouse | 2 weeks |

immunization

ELLSA tail blood potency testing

| | | | | |
|---|---|---|---|---|
| Shock | Intraperitoneal, | — | 50 | 3 days before |
| immunization | tail vein, or | | | the fusion |
| | intrasplenic | | | |

### Example 3: Subtype Analysis and Potency Testing of Monoclonal Antibody

Subtype analysis of monoclonal antibody: the subtypes of the antibodies PS-8 and PS-17 generated by 2 hybridoma cell lines secreting specific monoclonal antibodies are identified, respectively. The results show that PS-8 is IgG2b, and PS-17 is IgG1.

Potency testing of monoclonal antibody: a plate was coated with 1 μg/ml PS-KLH, each antibody was subjected to gradient dilution (1:100, 1:500, 1:2500, 1:12500, 1:32500 and 1:612500), and goat anti-mouse IgG-HRP was added to undergo 1w-fold dilution to ensure the potency of the purified monoclonal antibody (the results are as shown in Table 2).

TABLE 2
PS-BSA mouse five-immune tail blood potency testing
Coat the plate: PS-KLH 1 μg/ml
Second antibody: goat anti-mouse IgG-HRP 1w-fold
dilution

| | Immunized | Immunized | Immunized |
|---|---|---|---|
| Dilution | mouse-1 | mouse-2 | mouse-3 |
| fold | 1 | 2 | 3 |
| 100 | (+) | (+) | (+) |
| 500 | (+) | (+) | (+) |
| 2500 | (+) | (+) | (+) |
| 12500 | 2.235 | 3.417 | 3.125 |
| 32500 | 0.712 | 0.912 | 0.872 |
| 612500 | 0.268 | 0.425 | 0.428 |
| PBS | 0.098 | 0.103 | 0.065 |

### Example 4: Screening and Identification of Monoclonal Antibody

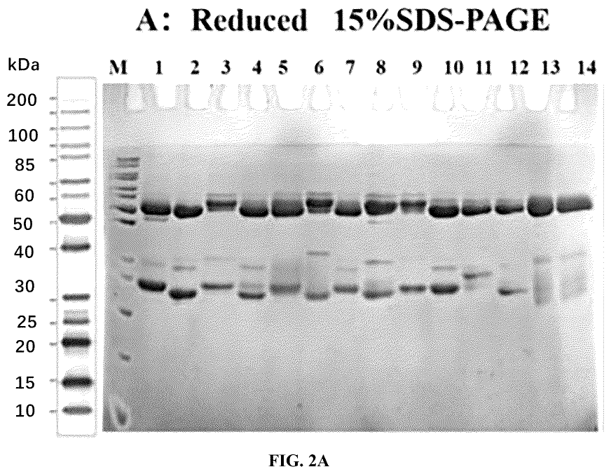

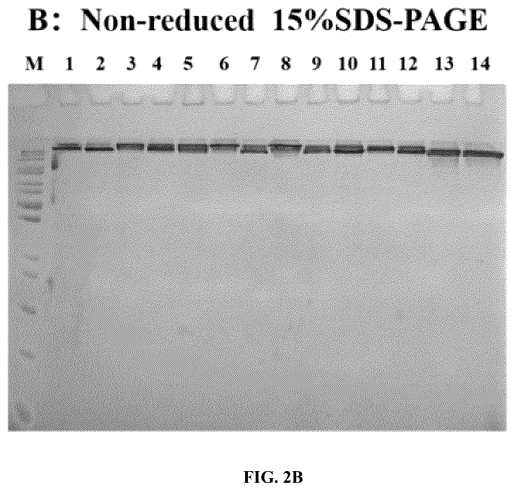

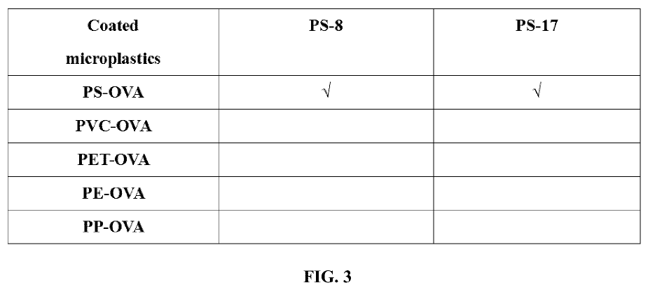

PS-BSA immunized mice were subjected to cell fusion, 12 positive hybridoma cells were obtained through 4 rounds of subcloning, monoclonal antibodies were prepared using the 12 positive hybridoma cells according to the above-mentioned method, and each antibody was subjected to reduced and non-reduced SDS-PAGE electrophoresis detection (the results are as shown in FIG. 2). Meanwhile, a control experiment was conducted on each antibody which was coated with 5 different types of microplastics including PS, polyvinyl chloride (PVC), polyethylene terephthalate (PET), PE and PP (the results are shown in FIG. 3) to confirm that there was no cross-reaction between the PS monoclonal antibody and other types of micro-nano plastics.

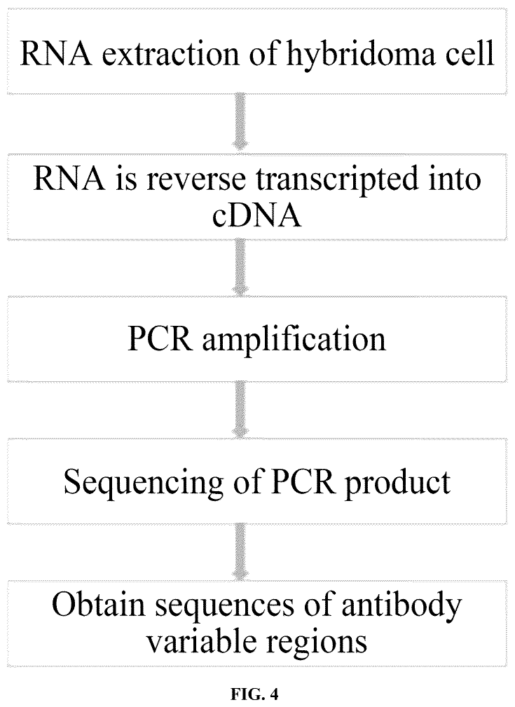

### Example 5: Sequencing of Monoclonal Antibody (The Sequences of the Variable Regions Of The Antibody Were Obtained According To The Procedure in FIG. 4)

(1) The monoclonal antibody secreted by the hybridoma cell line PS-8 comprises a heavy chain variable region and a light chain variable region both of which are composed of determining cluster complementarity regions and framework regions; the determining cluster complementarity regions of the heavy chain variable region and the light chain variable region are both composed of cerebellar degeneration related 1 (CDR1), CDR2, and CDR3;

The amino acid sequence of CDR1 of the heavy chain variable region is as shown in position 50-54 of SEQ ID NO.1;

The amino acid sequence of CDR2 of the heavy chain variable region is as shown in position 69-85 of SEQ ID NO.1;

The amino acid sequence of CDR3 of the heavy chain variable region is as shown in position 118-127 of SEQ ID NO.1;

The amino acid sequence of CDR1 of the light chain variable region is as shown in position 44-54 of SEQ ID NO.2;

The amino acid sequence of CDR2 of the light chain variable region is as shown in position 70-76 of SEQ ID NO.2;

The amino acid sequence of CDR3 of the light chain variable region is as shown in position 109-117 of SEQ ID NO.2.

The amino acid sequence of the heavy chain variable region of the PS-8 antibody is as shown in SEQ ID NO.1:

MEWSWIFLFLLSGTAGVHSEVQLQQSGPELVKPGASVKMSCKASGY
TFTSYVMHWVKQKPGQGLEWIGYINPYNDGTKYNEKFKGKATLTSD
KSSSTAYMELSSLTSEDSAVYYCASSYYYGSSYGYWGQGTTLTVSS.

The amino acid sequence of the light chain variable region of the PS-8 antibody is as shown in SEQ ID NO.2:

MESQIQVFVFVFLWLSGVDGDIVMTQSHKFMSTSVGDRVSITCKAS
QDVSTAVAWYQQKPGQSPKLLIYSASYRYTGVPDRFTGSGSGTDFT
FTISSVQAEDLAVYYCQQHYSTPLTFGAGTKLELK.

(2) The monoclonal antibody secreted by the hybridoma cell line PS-17 comprises a heavy chain variable region and a light chain variable region both of which are composed of determining cluster complementarity regions and framework regions; the determining cluster complementarity regions of the heavy chain variable region and the light chain variable region are both composed of CDR1, CDR2 and CDR3;

The amino acid sequence of CDR1 of the heavy chain variable region is as shown in position 50-54 of SEQ ID NO.3;

The amino acid sequence of CDR2 of the heavy chain variable region is as shown in position 69-85 of SEQ ID NO.3;

The amino acid sequence of CDR3 of the heavy chain variable region is as shown in position 118-128 of SEQ ID NO.3;

The amino acid sequence of CDR1 of the light chain variable region is as shown in position 44-54 of SEQ ID NO.4;

The amino acid sequence of CDR2 of the light chain variable region is as shown in position 70-76 of SEQ ID NO.4;

The amino acid sequence of CDR3 of the light chain variable region is as shown in position 109-117 of SEQ ID NO.4.

The amino acid sequence of the heavy chain variable region of the PS-17 antibody is as shown in SEQ ID NO.3:

MEWIWIFLFILSGTAGVHSQVQLQQSGAELARPGASVKLSCKASGY
TFTDYYINWVKQRTGQGLEWIGEIYPGSGNTYYNEKFKGKATLTAD
KSSSTAYMQLSSLTSEDSAVYFCARSEIYGIYYFDYWGQGTTLTVS
S.

The amino acid sequence of the light chain variable region of the PS-17 antibody is as shown in SEQ ID NO.4:

METHSQVFVYMLLWLSGVEGDIVMTQSHKFMSTSVGDRVSITCKA
SQDVGTAVAWYQQKPGQSPKLLIYWASTRHTGVPDRFTGSGSGTD
FTLTISNVQSEDLADYFCQQYSSYPYTFGGGTKLEIK.

### Example 6: Use of Monoclonal Antibody

Water and soil samples were collected from oceans, rivers and lakes. Textile wastewater samples were collected from textile enterprises. Food samples were collected from fields or dining tables. The collected samples were subjected to pretreatment and extraction, followed by quantitative detection using a reagent kit.

The pretreatment was conducted according to the following steps:

(1) Each of the liquid samples such as water samples was centrifuged for 3 min at 17000×g, a supernatant was digested and extracted, and the digested extracting solution was diluted using a sample diluent for test strip immunochromatography quantitative detection.

(2) Non-liquid samples were subjected to immunochromatographic quantitative detection after digestion treatment, microplastic extraction and diluent dilution.

A soil sample (Label testing) was taken as an example:

Deionized water was added into the soil sample (1:1 w/v) and then subjected to vortex for 10 s. A plastic mixture was centrifuged for 3 min at 17000×g, and the supernatant was used as a specific matrix extract for labeling recovery analysis. Sample detection was performed according to the following steps:

1. 50 μL of sample pretreatment extracting solution was added into an aseptic glass tube (parallel repeated samples were set during the sample testing), and meanwhile different volumes of microplastic working solutions were added in the same batch of tubes, the final concentrations of the added standard working solutions were 0, 1.57 μg, 3.125 μg, 6.25 μg, 12.5 μg, 25 μg and 50 μg respectively, and therefore standard curves for sample detection were established.

2. 950 μL of PBST washing buffer was then added to each tube.

3. The tubes were placed on a rotator to be rotated for 3 min at the speed of 20 r/min.

4. The tube was centrifuged for 3 min at the rotary speed of 5500 rpm to precipitate microplastic particles. The supernatant was carefully removed and discarded.

5. 1,000 μL of antibody diluent (32500-fold dilution) sample was added into each tube. The tube was subjected to a transient vortex or moved several times using a pipette to ensure the microplastics were completely mixed with the antibody solution.

6. The tube rotated for 1 h at the speed of 20 r/min at a room temperature.

7. Washing step. The tube was centrifuged for 3 min at the rotary speed of 5500 rpm to precipitate microplastic particles. The supernatant was carefully removed and discarded. 1,000 μL of washing buffer was added into each tube and the tube was subjected to transient vortex. The tube was placed on a vortex instrument to be rotated for 3 min at the rotary speed of 20 rpm.

8. Step 7 was repeated 3 times.

9. After the last washing step was completed, the tube was centrifuged for 3 min at 5500 rpm to precipitate microplastic particles. The supernatant was carefully removed and discarded.

10. 1,000 μL of goat anti-rabbit antibody labeled with horseradish peroxidase, which had been diluted 2,000 times with a PBST solution, was added into each tube. The tube was subjected to vortex for 1 min to ensure that the microplastics were completely mixed with the second antibody.

11. The tube was placed on the vortex instrument to be rotated for 1 h at 20 rpm at a room temperature.

12. Microplastics were washed 4 times using a washing buffer (steps 8-10 were repeated).

13. 100 μL of tetramethylbenzidine (TMB) substrate was added and moved several times up and down using a pipette, and then the suspension was instantly transferred to the wells of a microplate.

14. The microplate was placed at room temperature for 30 min of slow shaking culture.

15. 100 μL of 1M H₂SO₄ was added into each well for terminating the reaction.

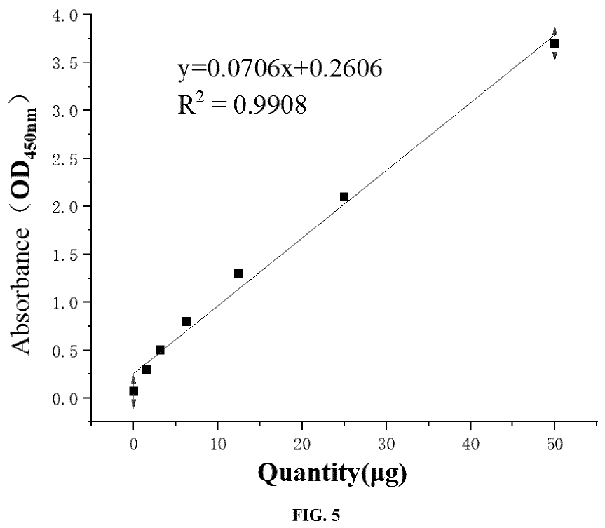

16. The optical density (OD) value was measured at 450 nm using a microplate reader; a standard curve was plotted using Origin (FIG. 5) based on the concentration of each standard substance as a horizontal coordinate and B/BO as a longitudinal coordinate.

17. The OD value of the detected sample was substituted into the standard curve to be calculated to obtain the amount of the microplastics in the sample.

Finally, it should be noted that the above descriptions are only preferred embodiments of the present disclosure, but are not used for limiting the present disclosure. Although the present disclosure has been illustrated in detail by referring to the foregoing embodiments, those skilled in the art still make modifications to the technical solutions described in the foregoing embodiments, or equivalent replacements on a part of technical features. Any modifications, equivalent replacements, improvements and the like made within the spirit and principle of the present disclosure should be included within the scope of protection of the present disclosure.

What is claimed is:

1. A hybridoma cell line secreting an anti-polystyrene monoclonal antibody, comprising hybridoma cell line PS-8 deposited with China Center for Type Culture Collection under the Accession number of CCTCC NO: C2024144.

2. A hybridoma cell line secreting an anti-polystyrene monoclonal antibody, comprising hybridoma cell line PS-17 deposited with China Center for Type Culture Collection under the Accession number of CCTCC NO: C2024154.

3. A monoclonal antibody, the monoclonal antibody is secreted by the hybridoma cell line PS-17 according to claim 2, and the monoclonal antibody specifically recognizes polystyrene.

4. A kit, comprising the monoclonal antibody secreted by the hybridoma cell line PS-8 or the hybridoma cell line PS-17 according to claim 3.

5. Use of the monoclonal antibody according to claim 3 in preparing a reagent for detecting polystyrene.


SEQUENCE LISTING (4 sequences)

SEQ ID NO: 1 - protein, 138 aa, synthetic construct
  source 1..138: note = the heavy chain variable region of the PS-8 antibody
MEWSWIFLFLLSGTAGVHSEVQLQQSGPELVKPGASVKMSCKASGYTFTSYVMHWVKQKPGQGLEWIGYINPYNDGTKYNEKFKGKATLTSDKSSSTAYMELSSLTSEDSAVYYCASSYYYGSSYGYWGQGTTLTVSS

SEQ ID NO: 2 - protein, 127 aa, synthetic construct
  source 1..127: note = the light chain variable region of the PS-8 antibody
MESQIQVFVFVFLWLSGVDGDIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPLTFGAGTKLELK

SEQ ID NO: 3 - protein, 139 aa, synthetic construct
  source 1..139: note = the heavy chain variable region of the PS-17 antibody
MEWIWIFLFILSGTAGVHSQVQLQQSGAELARPGASVKLSCKASGYTFTDYYINWVKQRTGQGLEWIGEIYPGSGNTYYNEKFKGKATLTADKSSSTAYMQLSSLTSEDSAVYFCARSEIYGIYYFDYWGQGTTLTVSS

SEQ ID NO: 4 - protein, 127 aa, synthetic construct
  source 1..127: note = the light chain variable region of the PS-17 antibody
METHSQVFVYMLLWLSGVEGDIVMTQSHKFMSTSVGDRVSITCKASQDVGTAVAWYQQKPGQSPKLLIYWASTRHTGVPDRFTGSGSGTDFTLTISNVQSEDLADYFCQQYSSYPYTFGGGTKLEIK